SEQUENCE LISTING (1 sequence)

SEQ ID NO: 1 - DNA, 212 bp, synthetic construct
  source 1..212: mol_type = other DNA
aattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaag

(19) United States
(12) Patent Application Publication
(10) Pub. No.: US 2025/0134905 A1
(43) Pub. Date: May 1, 2025

(54) USE OF SMALL-MOLECULE COMPOUND HOMOHARRINGTONINE IN MANUFACTURING DRUG FOR TREATING ACQUIRED IMMUNE DEFICIENCY SYNDROME

(71) Applicant: Hangzhou City University, Hangzhou (CN)

(72) Inventors: Geng YANG, Hangzhou (CN); Yashuang JIANG, Hangzhou (CN); Zhujiao ZHOU, Hangzhou (CN); Chong ZHANG, Hangzhou (CN); Jingyi YANG, Hangzhou (CN); Xiaolei PAN, Hangzhou (CN); Jiankang ZHANG, Hangzhou (CN); Jiajia WANG, Hangzhou (CN)

(73) Assignee: Hangzhou City University, Hangzhou (CN)

(21) Appl. No.: 18/917,049

(22) Filed: Oct. 16, 2024

Related U.S. Application Data
(63) Continuation of application No. PCT/CN2022/106150, filed on Jul. 18, 2022.

(30) Foreign Application Priority Data
Jul. 7, 2022 (CN) 202210791630.X

(51) Int. Cl. A61K 31/55

(52) U.S. Cl. CPC A61K 31/55

(57) ABSTRACT

The present invention relates to use of homoharringtonine in manufacturing a drug for treating an acquired immunodeficiency syndrome. A homoharringtonine compound is prepared as a drug for treating the acquired immunodeficiency syndrome and is applied to the treatment of the acquired immunodeficiency syndrome. The homoharringtonine compound can significantly reduce the programmed ribosomal frameshifting (PRF) efficiency of the mRNA of HIV-1 during the translation of a Gag-Pol fusion protein to substantially decrease the translation efficiency of the Pol protein. Therefore, the cell fails to produce protease, integrase, and reverse transcriptase obtained via Pol splicing such that a virus cannot be packaged effectively, which eventually leads to a sharp decline in yield of the virus. Therefore, the compound of the present invention has a good anti-HIV effect.

### CROSS-REFERENCE TO RELATED APPLICATIONS

The application claims priority to Chinese patent application No. 202210791630.X, filed on Jul. 7, 2022, the entire contents of which are incorporated herein by reference.

### SEQUENCE LISTING

The sequence listing xml file submitted herewith, named BPUS011240029.xml”, created on Dec. 5, 2024, and having a file size of 2,250 bytes, is incorporated by reference herein.

### TECHNICAL FIELD

The present invention relates to use of a homoharringtonine compound in manufacturing a drug for treating acquired immune deficiency syndrome (AIDS).

### BACKGROUND

Lots of retroviruses including human immunodeficiency virus-1 (HIV-1) and some coronaviruses such as severe acute respiratory syndrome and infectious bronchitis virus utilize programmed-1 ribosomal frameshifting (−1PRF) to control the production proportion of the structural protein to zymoprotein of the virus, which is beneficial to the assembly of the virus itself.

The −1PRF signal in HIV is located between open reading frames (ORFs) gag and pol; ribosome can identify the region and can induce the signal to shift backward a nucleotide within the region, thereby translating into a Gag-Pol fusion protein; the protein Gag is the structural protein of the virus, and the Pol is the key zymoprotein of the virus. The specific mechanism is as follows: during the translation process of Gag-pol mRNA, most of the ribosomes terminate at the termination codon of the ends of the gag ORF, to generate Gag protein only.

However, since the gag-pol mRNA of HIV-1 contains a stem loop consisting of a seven-nucleotide slippery sequence (U UUU UUA) and a downstream RNA stem loop, when a ribosome encounters the signal in the translation process, the ribosome will suspend in the sequence, and about 5% ribosome can be approximately induced to shift backward a nucleotide, which thus skips the termination codon of the Gag protein and also enables the subsequent ORF sequence to be transformed into the coding sequence of Pol protein, thereby producing the Gag-Pol fusion protein. Under the further action of the viral protease, the Gag protein is sliced into structural proteins such as p17, p24, and p7, and the Pol protein is sliced into zymoproteins such as reverse transcriptase, and Integrasse within the fusion protein. Just employing such a mechanism, HIV strictly controls the production proportion of the Gag-Pol protein to be 20:1, beneficial to the assembly of the virus itself.

Great progress has been achieved in the treatment of anti-retrovirus at present, but an HIV-1-induced AIDS is still a global health issue. Such a situation makes the development of a novel anti-HIV-1 drug and the research of a novel therapeutic target against the virus urgent. The frameshifting efficiency of HIV-1 controls the proportion of Gag: Pol; the deviation of the proportion, in particular to, the decrease of the frameshifting efficiency will lead to the reduced content of the Pol protein such that the cell fails to produce the zymoprotein required by the virus. Therefore, changing the −1PRF efficiency can be a novel anti-HIV-1 target. The inventor of the present patent just makes use of the HIV-1 mechanism to screen out a compound capable of significantly inhibiting HIV −1PRF from more than 2000 drugs approved to be listed by the Food and Drug Administration (FDA), thereby obtaining a potential drug capable of treating HIV-1.

### SUMMARY

The objective of the present invention is to provide use of a homoharringtonine compound in manufacturing a drug for treating an AIDS. Currently, the drug is merely directed against leukemia in the domestic and FDA-approved indications. It has been found by the applicants that the drug can be used for the treatment of HIV-1. There is no granted patent or literature to indicate that homoharringtonine can be used for the treatment of HIV-1. This is our brand-new invention. Moreover, the present invention is not directly correlated to the first use of the drug, and thus is a novel indication. Therefore, the patent inventor applies for a second patent of medical use for the medicament.

To achieve the above objective, the present invention adopts the following technical solutions:

Firstly, a dual-luciferase reporter gene is constructed, as shown in the figure below:

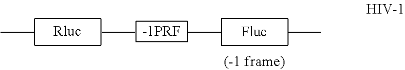

As can be seen from the figure above, the −1PRF sequence derived from HIV-1 is inserted into between coding sequences of a renilla luciferase (Rluc) and a firefly luciferase (Fluc). As such, Rluc can be transcribed and translated normally and thus, can be used as an internal reference, while FLuc can be translated only when the ribosome generates a −1 displacement code within the HIV-1 frameshifting signal zone. Therefore, we can utilize the change of Fluc/Rluc ratio to judge the variation of the −1PRF efficiency.

In the present invention, the sequence is constructed onto a pLVX-neomycin plasmid, and then packaged as a virus together with pspAX2 and pMD2G; the virus is infected to cells Hela and 293 to construct a stable cell line by G418 screening, thus achieving large-scale drug screening.

Approximate 2000 drugs approved to be listed by the FDA are adopted in the present invention as the dual-luciferase cell lines; when the ratio of the Fluc/Rluc reduces significantly, it indicates that the drug has an inhibiting effect on the −1PRF efficiency; hence, a novel target and drug can be screened out. By such a method, the compound homoharringtonine obtained in the present invention can significantly reduce the ratio of Fluc/Rluc in model cells, and is further demonstrated to have a significant inhibiting effect on the production of HIV-1 virus at a cellular level.

The present invention has the following technical points:
(1) −1PRF length and sequence: the present invention adopts the following HIV −1PRF sequence having a length of 212 bp including a slippery sequence and a stem-loop structure: 5′-AA T TTT TTA GGGAAG ATCTGGCCTTCCCACAAGGGAAGGCCAGGGAATTTTCTTCAGAGCAGACCAGAGCCA ACAGCCCCACCAGAAGAGAGCTTCAGGTTTGGGGAAGAGACAACAACTCCCTCTCA GAAGCAGGAGCCGATAGACAAGGAACTGTATCCTTTAGCTTCCCTCAGATCACTCTTT GGCAGCGACCCCTCGTCACAATAAAG-3′, (as shown in SEQ ID No. 1) where the black bold sequences denote the slippery sequence and the stem-loop structure; the underlined sequence denotes the position of the termination codon in absence of −1PRF, that is, the cell only produces protein Rennila. The TAA termination codon appears frameshifting only when −1PRF occurs, thereby translating the downstream Luciferase protein.
(2) Drug bank information: we purchased a drug bank approved to be listed by FDA. The drug bank contains 1971 different small-molecule compounds, as shown in FIG. 1. The inventor treated the pLVX-Rennila-(−1PRF)-Luciferase model cell lines constructed in this laboratory by these small-molecule compounds one by one to calculate the change of the Fluc/Rluc ratios after the treatment of different drugs, screen out the potential small-molecule compounds with a declined Fluc/Rluc ratio, and finally to obtain 4 small-molecule compounds capable of highly significantly inhibiting the HIV −1PRF efficiency through different concentration treatment and multiple repeated experiments. The screening positive rate is 2/1000, one of them is homoharringtonine. The chemical structural formula is shown in FIG. 2. Homoharringtonine is a kind of alkaloid extracted from branches, leaves, and bark of Cephalotaxus fortunei peculiar in southern China. China has initiatively applied homoharringtonine to acute myelogenous leukemia and chronic myelogenous leukemia since the 70s, and was approved by the FDA in 2012 to be listed in the USA. However, there is yet no granted patent and literature at present to indicate homoharringtonine can be used for the treatment of HIV-1.
(3) Homoharringtonine significantly decreases the HIV-1 −1PRF efficiency in stably-transfected cells Hela and 293; it acts on the cells Hela and 293 stably-transfected with Rennila-(−1PRF)-luciferase-by chemical dosing; Rennila and Luciferase values are detected by a multimode reader. The results are shown in FIG. 3: as compared to the control group, the cell treated with homoharringtonine has a significantly reduced Fluc/Rluc value.
(4) When the pLVX-Rennila-(−1PRF)-luciferase was transiently transfected into THP1 cells and normal human peripheral blood mononuclear macrophages, 48 hours later, the cells were treated with homoharringtonine; values Rennila and Luciferase were detected by a multimode reader. It was found that as shown in FIG. 4, the Fluc/Rluc value of the homoharringtonine-treated cells likewise decreased markedly.
(5) When we used different concentrations of homoharringtonine to treat the 293 cells stably transfected with Rennila-(−1PRF)-luciferase, as shown in FIG. 5, it was found that homoharringtonine could significantly decrease the −1PRF efficiency at 5 nM.
(6) Homoharringtonine is found to be used for the treatment of leukemia at present. Hence, we selected small-molecule compounds Fludarabine and Mitoxantrone which can be used for the treatment of leukemia in the drug bank to treat cells and to observe whether Fludarabine and Mitoxantrone affected the −1PRF mechanism of the HIV cell, thus affecting the production of HIV. As can be seen from FIG. 6, relative to the significantly decreased−1PRF efficiency in the treatment group of homoharringtonine, the Fludarabine and Mitoxantrone treatments fail to significantly decrease the −1PRF in cells, which indicates that Fludarabine and Mitoxantrone cannot act on the mechanism of HIV −1PRF and thus, cannot affect the packaging of the virus. Therefore, as can be seen from the result, the drug capable of treating leukemia can be not necessarily used for the treatment of HIV, there is no direct correlation.
(7) After the pNL4-3-Envolope mutant plasmid was transfected into 293 cells, the plasmid could package the HIV-1 particle with a mutant outer membrane protein; the HIV-1 particle was non-infectious, but could represent the condition of generating virus particles within cells and could be used for the measurement of EC50 of a drug. By such a method, we measured that the EC50 concentration of homoharringtonine in inhibiting the production of HIV-1 was 8.57 nM (FIG. 7).
(8) After 293T cells were transfected with the pNL4-3-Envolope mutant plasmid for 24 hours, we treated the cells with different concentrations of homoharringtonine for 12 hours and collected the cells, then detected the expression of the Gag-Pol fusion protein in the cells by Western blot. As shown in FIG. 8, when the drug concentration is up to 20 nM, the Gag-Pol fusion protein is basically undetectable.
(9) Besides the −1PRF sequence of HIV, we also constructed the −1PRF sequence of three viruses SIV, RSV, and MMTV between Rennila and Lucifease. These plasmids were transiently transfected into 293 cells for 48 hours, and then the cells were treated with homoharringtonine. The compound is found to also reduce the −1PRF efficiency of different viruses (FIG. 9), which indicates that the drug is broad-spectrum in inhibiting the −1PRF efficiency of virus and homoharringtonine is a potential therapeutic drug as long as the virus has the −1PRF mechanism.
(10) We obtained immunodeficient mouse NOD.Cg-Prkdcscid Il2rgtm1Wj1/SzJ (NSG). Human hemopoietic stem cells were isolated and then transplanted into the mouse, and continuously cultured, two weeks later, the differentiation of the hemopoietic stem cells in the mouse in vivo was detected, to finally obtain the mouse humanized by hematopoietic cells. Therefore, the patent inventor makes use of this model to evaluate the interaction effect of the drug in vivo. We purified the HIV and injected the virus into the blood system of the mouse via caudal vein, 48 hours later, we also injected homoharringtonine into the mouse via caudal vein. 48 hours later, we detected the content of the HIV particle in mouse blood. Results of FIG. 10 indicate that the HIV particle in the blood of the mouse injected with homoharringtonine is also significantly lower than that in the control group. The result indicates that in animal experiments, homoharringtonine may also inhibit the replication of HIV.

The benefits of the present invention are mainly embodied in:
(1) The homoharringtonine screened in the present invention can effectively reduce the occurrence of HIV −1PRF and can significantly inhibit the replication of the HIV-1 virus in model cells, THP1 cells, and peripheral blood mononuclear cells, thus inhibiting the assembly of the HIV-1 virus particle; (2) the present invention finds a novel indication of a leukemia therapeutic drug, i.e., homoharringtonine, that is, homoharringtonine has an anti-HIV-1 virus effect. Because the drug has appeared on the market, the present invention can significantly decrease the capital investment in safety evaluation of the drug in the early stage of research and development, and can reduce the research and development risk of the drug. (3) It has been found in the present invention that the drug is broad-spectrum in inhibiting −1PRF, and homoharringtonine is a potential therapeutic drug as long as the virus has the −1PRF mechanism.

### BRIEF DESCRIPTION OF DRAWINGS

FIG. 1 shows information such as quantity, type version, and stability of the drugs contained in the drug bank approved by FDA;

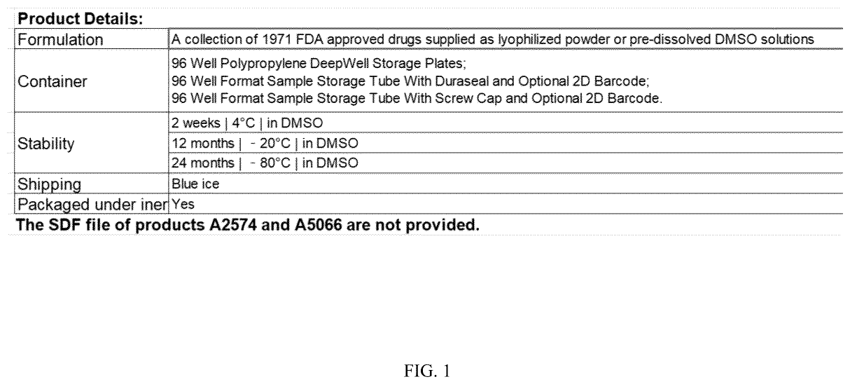

FIG. 2 shows a chemical structural formula of Homoharringtonine;

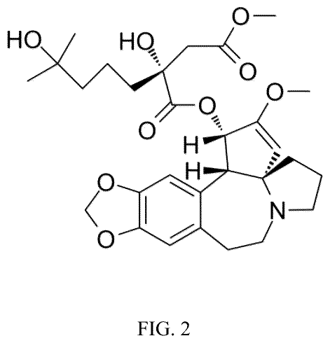

FIG. 3 shows changes in a Rennila/Luciferase ratio after cells Hela and 293 stably transfected with Rennila-(−1PRF)-luciferase are treated for 6 hours;

FIG. 4 shows changes in a Rennila/Luciferase ratio after THP1 and human peripheral blood mononuclear cells are transiently transfected with a plasmid pLVX-Rennila-(−1PRF)-luciferase and then treated by homoharringtonine for 6 hours;

FIG. 5 shows changes in a Rennila/Luciferase ratio after Rennila-(−1PRF)-luciferase stably-transfected cells are treated by different concentrations of homoharringtonine;

FIG. 6 shows changes in a Rennila/Luciferase ratio after the Rennila-(−1PRF)-luciferase stably-transfected cells are treated by Fludarabine, Mitoxantrone, and homoharringtonine;

FIG. 7 shows that peripheral blood mononuclear cells are transfected with a pNL43 envelop mutant plasmid for 24 hours and then treated with the addition of homoharringtonine for 6 hours, and the change of virus particles in medium supernatant is detected by p24 ELISA, to calculate EC50;

FIG. 8 shows that peripheral blood mononuclear cells are transfected with a pNL43 envelop mutant plasmid for 24 hours and then treated with the addition of homoharringtonine for 12 hours, and the expression of a Gag-Pol fusion protein in cells is detected by Western blot;

FIG. 9 shows changes in a Rennila/Luciferase ratio after cells are transiently transfected with the Rennila-(−1PRF)-luciferase plasmid of SIV, SARS-Covid19, RSV, and MMTV, and then treated with homoharringtonine for 6 hours; and

FIG. 10 shows detection of a change of HIV generation in virus of mouse blood after being treated by homoharringtonine in animal experiments.

### DETAILED DESCRIPTION OF THE EMBODIMENTS

The technical solution of the present invention will be further described in detail with reference to the accompanying drawings and detailed examples below.

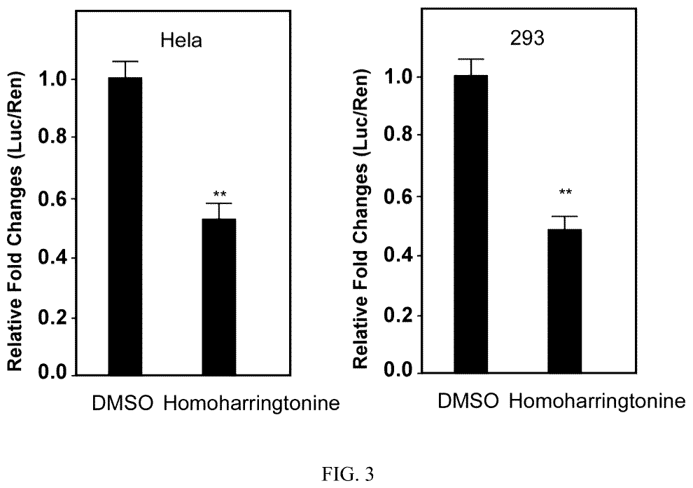

Example 1: as shown in FIG. 3, provided is use of a homoharringtonine compound in manufacturing a medicament for treating AIDS; the homoharringtonine compound is used for reducing the HIV −1PRF efficiency such that the Pol protein content declines and the virus may not be packaged as a complete particle in cells, thereby leading to a sharp decline in yield of the AIDS. Just utilizing such a mechanism of HIV, the present invention may screen out a compound capable of significantly inhibiting HIV −1PRF from the drug bank approved by FDA, thereby obtaining the potential drug capable of treating HIV.

In the present invention, Rennila-(−1PRF)-luciferase hela and Rennila-(−1PRF)-luciferase 293 stable cell lines capable of indicating the −1PRF efficiency may be constructed firstly, thus screening out a compound homoharringtonine capable of significantly reducing the −1PRF efficiency from more than 2000 drugs approved to be listed by FDA, and proving the inhibition of the compound in the −1PRF efficiency of HIV at a level of a stable cell line.

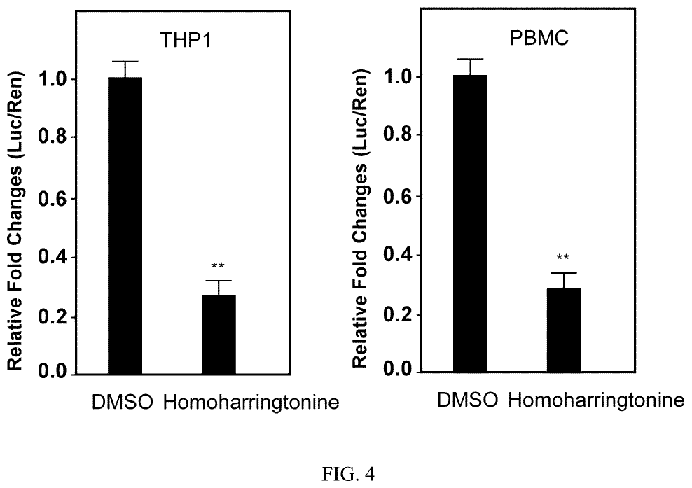

Example 2: as shown in FIG. 4, when homoharringtonine was applied, the constructed pLVX-Rennila-(−1PRF)-luciferase was transiently transfected into cells THP1 and PBMC, and then homoharringtonine was added to act on the cells; the changes of the Renilla and Luciferase expression quantity were detected with a multimode reader. The results show that the ratio of Renilla to Luciferase decreases significantly after the treatment of homoharringtonine.

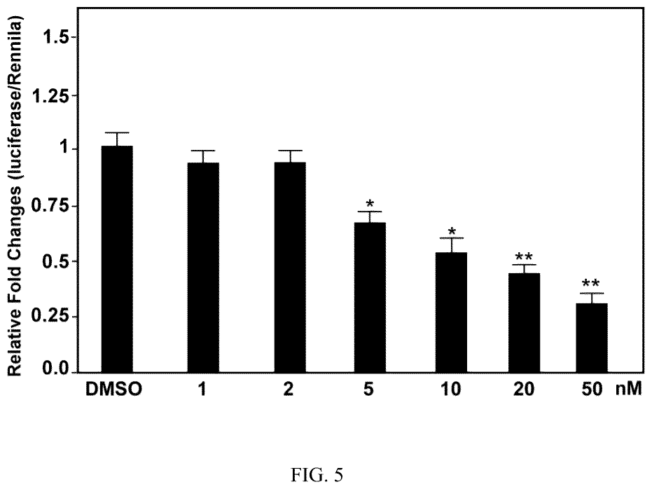

Example 3: please referring to FIG. 5, based on Example 1, the stably-transfected cells pLVX-Rennila-(−1PRF)-luciferase 293 were treated by different concentrations of compound homoharringtonine and changes of the fluorescence values were measured. Results show that when the inhibition concentration of homoharringtonine is up to 5 nM, the ratio of Renilla to Luciferase decreases significantly; the expression results show that homoharringtonine may significantly inhibit the HIV −1PRF efficiency at such a concentration.

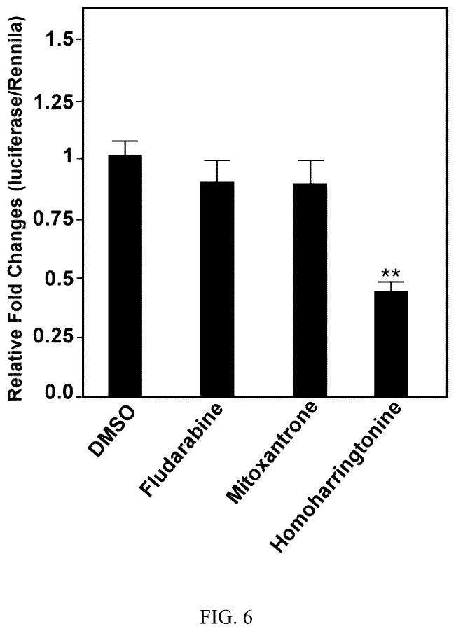

Example 4: please referring to FIG. 6, based on Example 1, three small-molecule compounds capable of treating leukemia, i.e., Fludarabine, Mitoxantrone, and Homoharringtonine were utilized to treat the stably-transfected cells pLVX-Rennila-(−1PRF)-luciferase 293 and changes of the fluorescence values were measured. The results show that−1PRF of the cells does not decrease significantly after being treated with Fludarabine and Mitoxantrone except homoharringtonine.

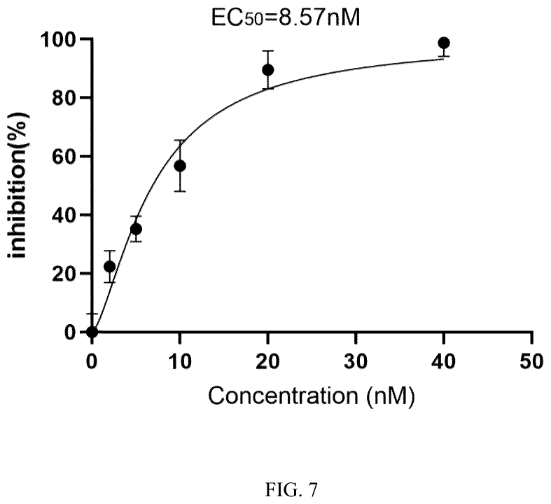

Example 5: please referring to FIG. 7, human embryo kidney 293 cells were transfected with a pNL4-3 envelope mutant plasmid for 24 hours, and then treated with different concentrations of homoharringtonine for 12 hours; the change of virus particles in a medium was detected by p24 ELISA; absorbance was detected by a spectrophotometer at OD450, and EC50 of the compound homoharringtonine was calculated. The results show that the EC50 of the compound homoharringtonine is 8.57 nM.

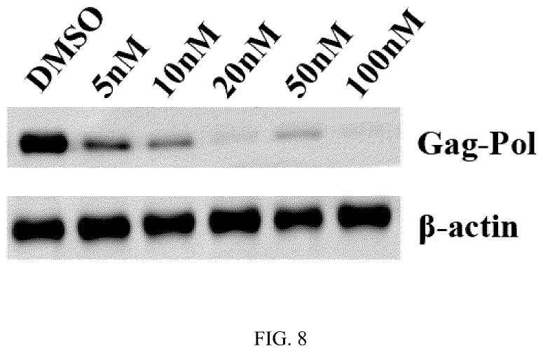

Example 6: please referring to FIG. 8, embryo kidney 293 cells were transfected with a pNL4-3 envelope mutant plasmid for 24 hours and then treated with different concentrations of homoharringtonine compound for 12 hours, and collected; the collected cells were lysed, then the expression of the Gag-Pol fusion protein in cells was detected by Western blot. The results show that when the compound homoharringtonine is 5 nM, the content of Gag-Pol in cells has decreased significantly, when the compound homoharringtonine is up to 20 nM, the Gag-Pol fusion protein is basically undetectable in cells.

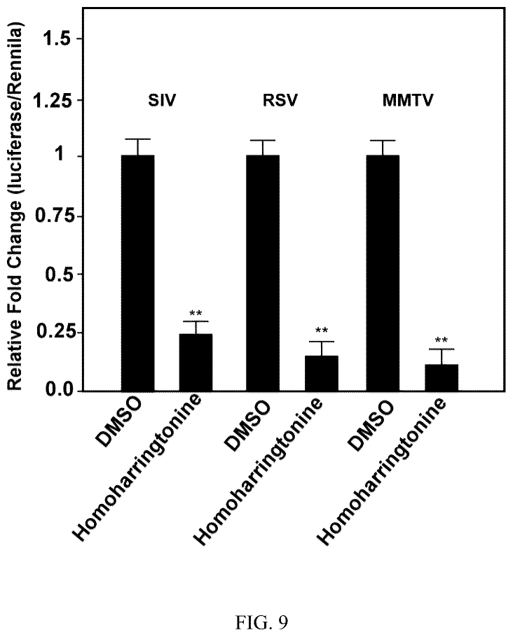

Example 7: please referring to FIG. 9, besides the −1PRF sequence of HIV, we also constructed the −1PRF sequence of four viruses SIV, SARS-COVID19, RSV, and MMTV between Rennila and Lucifease, respectively. After the 293 cells were transiently transfected with these plasmids for 48 hours, and then homoharringtonine was added to act on the cells. The changes in the expression quantity of Renilla and Luciferase were detected by a multimode reader. The results show that the ratio of Renilla to Luciferase in the −1PRF sequences constructed via these different viruses decreases significantly after the treatment of homoharringtonine. The result indicates that the compound is broad-spectrum in inhibiting the −1PRF efficiency of the virus, and homoharringtonine is a potential therapeutic drug as long as the virus has the −1PRF mechanism.

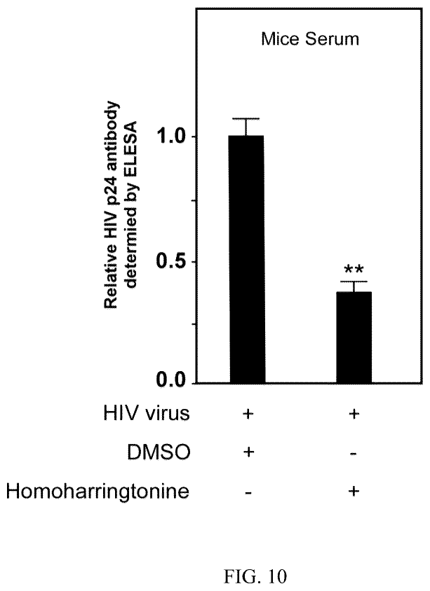

Example 8: please referring to FIG. 10, human hemopoietic stem cells were isolated and then transplanted into the immunodeficient mouse NOD.Cg-Prkdcscid Il2rgtm1Wj1/Example (NSG), two weeks later, a blood cell-humanized mouse animal model was established. The HIV was purified and injected into the blood system of the mouse via caudal vein, 48 hours later, we then injected homoharringtonine into the mouse via caudal vein. 48 hours later, the patent inventor detected the content of the HIV particle in mouse blood by ELISA. As can be seen from the figure, homoharringtonine may also significantly inhibit the replication of HIV in the humanized mouse blood.

In conclusion, the beneficial effects of the present invention are mainly embodied as follows: (1) the homoharringtonine screened in the present invention may effectively reduce the occurrence of HIV −1PRF and may significantly inhibit the replication of the HIV-1 virus in model cells, THP1 cells, and peripheral blood mononuclear cells, thus inhibiting the assembly of the HIV-1 virus particle; (2) the present invention finds a novel indication of a leukemia therapeutic drug, i.e., homoharringtonine, that is, homoharringtonine has an anti-HIV-1 virus effect. Because the drug has appeared on the market, the present invention may significantly decrease the capital investment in safety evaluation of the drug in the early stage of research and development, and can reduce the research and development risk of the drug. (3) It is found in the present invention that the drug is broad-spectrum in inhibiting−1PRF, and homoharringtonine is a potential therapeutic drug as long as the virus has the −1PRF mechanism.

The detailed embodiments mentioned above are to further specify the objectives, technical solutions and beneficial effects of the present invention. It should be understood that what is described above are merely detailed embodiments of the present invention, but are not construed as limiting the protection scope of the present invention. Any amendment, equivalent replacement, improvement, and the like made within the spirit and principle of the present invention shall fall within the protection scope of the present invention.

What is claimed is:

1. Use of a homoharringtonine compound in manufacturing a drug for treating an acquired immunodeficiency syndrome, wherein the homoharringtonine compound is prepared as a drug for treating the acquired immunodeficiency syndrome.
